(19) United States
(12) Patent Application Publication
(10) Pub. No.: US 2008/0241853 A1
(43) Pub. Date: Oct. 2, 2008

(54) MICROTITER SPIN ARRAY

(75) Inventors: CHRISTIAN L. SCHOEN, Laramie, WY (US); SHELLEY J. COLDIRON, Salt Lake City, UT (US)

Correspondence Address: MCKEE, VOORHEES & SEASE, P.L.C., 801 GRAND AVENUE, SUITE 3200, DES MOINES, IA, 50309-2721, US

(73) Assignee: CONCURRENT ANALYTICAL, INC., Salt Lake City, UT (US)

(21) Appl. No.: 12/056,977

(22) Filed: Mar. 27, 2008

Related U.S. Application Data
(60) Provisional application No. 60/908,811, filed on Mar. 29, 2007.

(51) Int. Cl. G01N 33/53

(52) U.S. Cl. 435/7.1

(57) ABSTRACT

An improvement in heterogeneous immunoassays in microtiter plates to significantly reduce assay time, from as much as 50% up to 90% of what used to be typical assay times. The improvement involves the rotation of the liquid in a microtiter plate and during incubation times for antigen capture and during incubation times for sample labeling. This is accomplished through the insertion of fluted cylindrical stirrers in each well, and the use of a conventional, commercially available, microtiter vortexer.

### CROSS-REFERENCE TO RELATED APPLICATIONS

This application claims priority under 35 U.S.C. §120 to provisional application Ser. No. 60/908,811 filed Mar. 29, 2007, herein incorporated by reference in its entirety.

### BACKGROUND OF THE INVENTION

Immunoassay tests hold an important niche in human and veterinary medicine, and in bioterrorism prevention. Even with the success and widespread use of these tests, improvements in sensitivity, specificity, speed, cost, and throughput remain critical needs. This invention seeks to provide improvements in the speed and sensitivity offered by many of the methodologies employed in heterogeneous assays in microtiter plates. Heterogeneous immunoassays require the delivery of antigen to a solid capture substrate, and typically rely on diffusion as the mode of mass transport. Though easily implemented, diffusion limited mass transfer often results in long incubation times because large biological targets (e.g., proteins, viruses, and bacteria) have small diffusion coefficients. This ligation is amplified for sandwich-type assays since a tagged antibody is needed in order to identify and quantify the surface-bound antigen. Various approaches have been investigated to increase the flux of the antigen or label as a means to reduce incubation time, capitalizing on the fact that antibody-antigen binding is often limited by mass transport rather than by binding kinetics (i.e., recognition rate). Electric fields, for example, have been used to drive the transport of charged species in DNA hybridization assays and in heterogeneous immunoassays. The combination of superparamagnetic labels and magnetic fields have also been shown to be an effective pathway to increase flux. Typical heterogeneous immunoassays involve two steps that are significantly time limited. The first of the two steps is sample incubation in order to bind or capture the antigen to the substrate as illustrated FIG. 1. The second step involves attaching labels to the antigen bound to the substrate in order to allow detection. In FIG. 1, this overall process is illustrated; sample 10, is incubated with the antigen 12 to form a sandwich composite 14. Thereafter, the sandwich composite 14 is reacted with a detection label 16 to form the detection composite 18. The two limiting steps involve the sample incubation 30 period 20 and the label incubation period 22.

In a typical process such as for example surface-enhanced Raman spectroscopy there is a twelve hour incubation for sample incubation 20 and a twelve hour sample incubation for the label incubation 22. This results in twenty four hours of incubation time for each assay! This extremely lengthy time period means significantly decreased economics for running these assays.

There are a variety of ways that have been explored in the past in order to decrease significantly test times and as well, to enhance detection. For example, in the past, rotation of an immunoassay has been used to enhance detection. (see Huet, “A heterogeneous immunoassay performed on a rotating carbon disk electrode with electrocatalytic detection”, J. of Immunological Methods, 135:33-41 (1990)). It is important to note however that Huet is not addressing decreased assaying time, but rather enhanced detection. Put another way, Huet involves spinning or rotation of the composite sample 18 during detection, saying nothing of what happens during the already completed typical 24 hour period of sample incubation and label incubation, 20, 22.

Utilizing the technique of this invention as hereinafter described, it is possible to reduce typical times to perform surface-enhanced Raman spectroscopy in microtiter plates from 24 hours to 25 minutes! This is demonstrated in the examples below.

Accordingly, it is a primary objective of the present invention to improve a process of performing heterogeneous immunoassays by dramatically cutting the time for each assay. The method and means of accomplishing this primary objective as well as others will become apparent from the detailed description of the inventions which follows hereinafter.

### BRIEF SUMMARY OF THE INVENTION

An improvement in heterogeneous immunoassays in microtiter plates to significantly reduce assay time, from as much as 50% up to 90% of what used to be typical assay times. The improvement involves the rotation of the liquid in a microtiter plate and during incubation times for antigen capture and during incubation times for sample labeling. This is accomplished through the insertion of fluted cylindrical stirrers in each well, and the use of a conventional, commercially available, microtiter vortexer.

### BRIEF DESCRIPTION OF THE DRAWINGS

FIG. 1 shows a typical set of steps in a heterogeneous immunoassay as primarily limited by incubation times for antigen and label attachment to the assay sandwich.

FIG. 2 is a schematic of the process of this invention as applied to a microtiter plate format.

FIG. 3 is a schematic of the fluted cylindrical stirrer.

FIG. 4 is a picture of a typical microtiter plate.

FIG. 5 is a picture of a typical microtiter plate vortex instrument (Eppendorf MixMate).

FIG. 6 is a schematic of a 96 well plate with attached bottom for Surface Enhanced Raman Applications. The plate substrate is constructed on a semi-rigid ˜3 mm polymeric plate upon which 200 nm Au is deposited in a magnetron sputterer on a plate that has been milled by 200 μm to create raised Au addresses commensurate with wells. The gasket creates an adequate seal for the clamped assembly allowing traditional robotics to prepare the samples as in a traditional 96 well format. Convective stirring is accomplished on a shaker table that creates miniature vortices in each well. An additional ‘lid’ may be used to minimize evaporation or ‘sloshing’.

FIG. 7 shows the data of Table 1 in graph form.

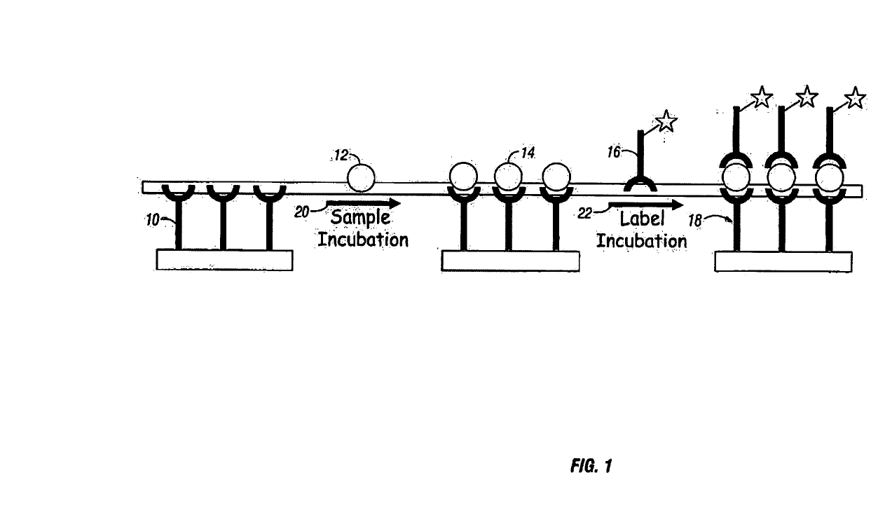

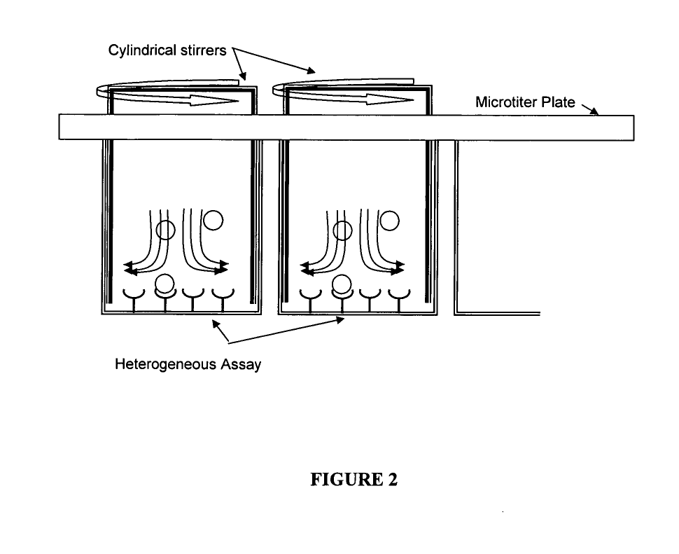

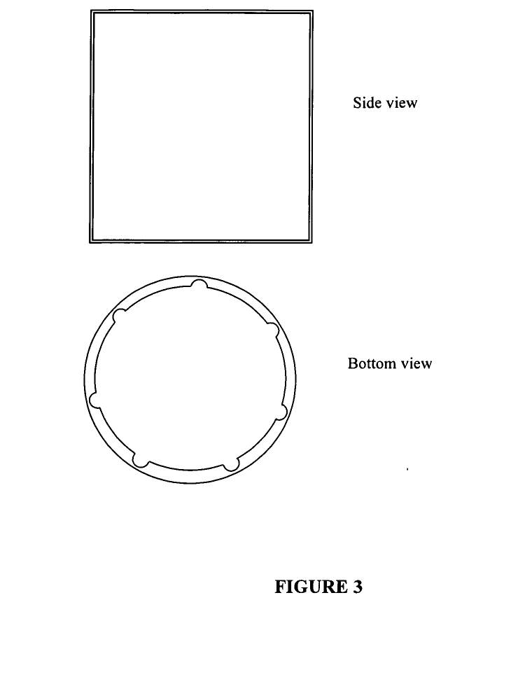

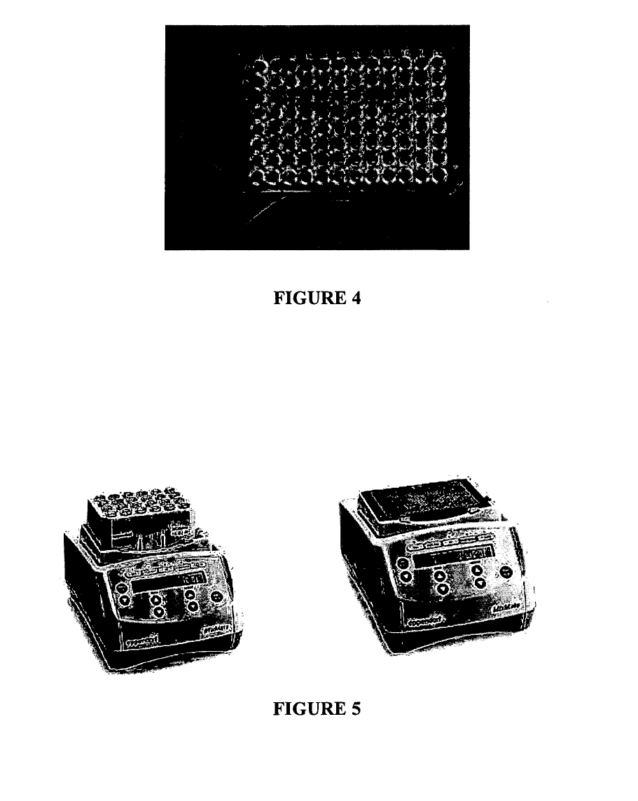

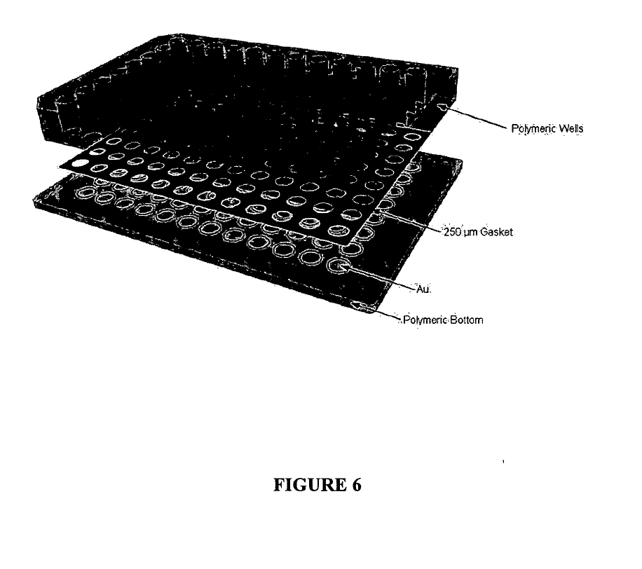

### DETAILED DESCRIPTION OF THE PREFERRED EMBODIMENT

This invention is an improvement to a previous invention described in patent application Ser. No. 11/677,464 entitled Spin Array, filed Feb. 21, 2007. In that invention, the substrate is rotated while the walls of the cell remain fixed. This combination creates the flux necessary to improve the heterogeneous assay times. In this invention, the substrate is the bottom of each well in a microtiter plate. The bottoms of conventional microtiter plates may not be rotated. To create the flux necessary to improve the heterogeneous assay times in each well of the microtiter plate, the liquid within the well must be rotated against the fixed bottom. This can be accomplished by inserting a cylindrical mixer in each well which is then rotated, or by using commercially available vortexing instruments. Vortexers, like the Eppendorf Mixmate have been conventionally used to mix small volumes in 96 and 384 well plates. By operating this vortexing instrument at speeds just below where the liquid vortexes, one can enhance the flux on the substrate well bottom thereby improving assay time. Description of the improvement to enhance flux in a heterogeneous assay is described in (Driskell, J. D.; Kwarta, K. M.; Lipert, R. J.; Porter, M. D.; Vorwald, A.; Neill, J. D.; Ridpath, J. F. J. Virol. Methods 2006, 138, 160-169).

$$\text{?} \\ \text{?} \text{indicates text missing or illegible when filed} \qquad ( 1 )$$

This article is of course not prior art against the invention and is mentioned here only for completeness. That paper examined the effectiveness of substrate rotation in the reduction of the time required for the antigen (i.e., virus) binding step. It also enumerated the captured viruses in a label free format by using force microscopy (AFM), noting that AFM is more readily applied in imaging objects the size of viruses but not of the proteins featured in the work herein. Moreover, the paper showed that the accumulation of bound antigen, represented by its surface concentration T., is given by Driskell, J. D.; Kwarta, K. M.; Lipert, R. J.; Porter, M. D.; Vorwald, A.; Neill, J. D.; Ridpath, J. F. J. Virol. Methods 2006, 138, 160-169) where D is the antigen diffusion coefficient, Cb is the bulk concentration of antigen, t is the incubation time, V is the kinematic viscosity of the solution, and o is the rotation rate. The first term on the right hand side of the equation represents the contribution of diffusional mass transfer, whereas the second term defines the role of substrate rotation on hydrodynamically-accelerated mass transfer. There are three assumptions assumed for the derivation of this equation. Driskell, J. D.; Kwarta, K. M.; Lipert, R. J.; Porter, M. D.; 10 Vonvald, A.; Neill, J. D.; Ridpath, J. F. J. Virol. Methods 2006, 138, 160-169). First, the reactant solution concentration is independent of binding. Second, the binding sites at the surface do not saturate. Third, the recognition reaction is first compared to the delivery of reactant. Equation 1 explicitly describes how antigen binding can be manipulated by varying t and, more importantly, o.

There are a few precedents for use of rotation in sandwich-type heterogeneous immunoassays. For example, Huet, supra, used rotation-controlled flux to devise an assay that was independent of sample volume. Other laboratories employed rotation in the amperometric detection step of an enzymatically generated redox probe. (Wijayawardhana, C. A.; Purushothama, S.; Cousino, M. A.; Halsall, H. B.; Heineman, W. R. J Electroanal. 20 Chem. 1999, 468, 2-8; Salinas, E., Tomero, A. A. J.; Sam, M. 1.; Battaglini, F.; Raba, J. Talanta 2005, 66, 92-102; Messina, G. A.; Torriero, A. A. J.; DeVito, I. E.; and Olsina, R. A,; Raba, J. Anal. Biochem. 2005, 337, 195-202). To our knowledge, this invention is the first to describe a rotation-based method designed to reduce both the antigen 20 and label binding 22 times. While the specific results and example herein described pertain to the use of a nanoparticle labeling scheme which exploits surface enhanced Raman scattering (SERS), the overall strategy can be applied to virtually any type of heterogeneous assay (e.g., scintillation counting, chemiluminescence, electrochemical, enzymatic methods, surface plasmon resonance, quantum dots, and microcantilevers.

As a proving ground for the merits of rotation in a sandwich immunoassay, the below described example uses a SERS-based labeling readout scheme previously developed by the inventors. This scheme uses extrinsic Raman labels (ERLs) to identify and quantify antigens in a sandwich immunoassay format. ERLs consist of gold nanoparticles that are coated with a layer of an intrinsically strong Raman scatterer that acts as a spectroscopic tag and a layer of an antibody that controls recognition specificity. Previous work resulted in the detection of only −60 binding events using 30-nm Ems, which translated to a limit of detection of −30 fM in an assay for prostate specific antigen in human serum. The inventors more recently reported on the detection of single-digit 15 binding events via larger (60 nm) ERLs, (Park, H.-Y.; Lipert, R. J.; Porter, M. D. Proc. SPIE 2004; 464-477), which optimized plasmon coupling with the underlying gold substrate at the laser excitation wavelength. (Park, H.-Y.; Lipert, R. J.; Porter, M. D. Proc. SPIE 2004; 464-477; and Driskell, J. D.; Lipert, R. J.; Porter, M. D. J. Phys. Chem. B 2006, 110, 17444-17451). While proving extremely sensitive, there are several challenges to advancing the scope of this readout strategy. One major obstacle rests with the long incubation times required by both the capture and labeling steps when under diffusion control. This complication is amplified by our assay format because the larger size of ERLs translates to lower diffusional mass transfer rates than those of more typical labels (e.g., fluorescently tagged antibodies). Estimates, which are based only on consideration of particle size via the Stokes-Einstein equation, (Berry, R. S.; Rice, S. A.; Ross, J. Physical Chemistry; John Wiley & Sons: New York, 1980) yield a diffusion coefficient for a 60-nm ERL that is roughly tenfold smaller than that of a fluorescently tagged antibody. Equation 1 therefore indicates that the labeling step with ERLs will be about three times slower than that for a fluorescently tagged antibody. By capitalizing on the second term in Equation 1, it should be possible to use substrate rotation to overcome the diffusion-based limitations to mass transfer in both the capture and labeling steps.

The following example is offered to illustrate but not limit the invention.

### EXAMPLE

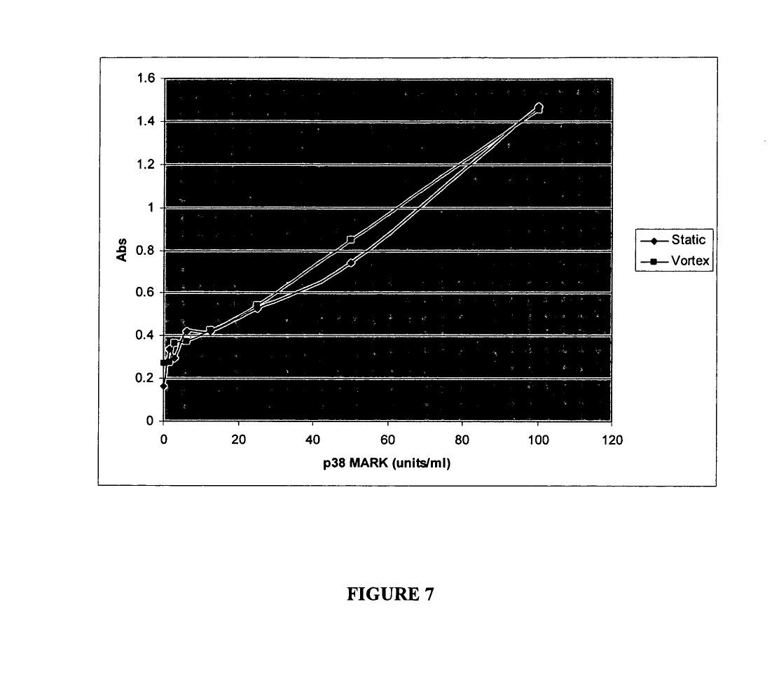

In order to test the efficacy of employing liquid rotation, applicants ran a test using a standard invitrogen Immunoassay Kit, Catalog #KH00071, p38 MAPK. The procedure was followed exactly as in the published protocol using the normal static procedure (no well rotation) and then run again with the only difference being this time vortexing was used. Table 1 discloses the date from these two runs, and FIG. 7 shows this same data in graph form, illustrating vortexing allows the procedure to be accomplished four times faster to achieve the same sensitivity.

| Step | Static (min) | Vertex (min) | Description |
|---|---|---|---|
| 1 | 120 | 30 | Incubate Standards |
| 2 | 60 | 15 | Incubate Detection Antibody |
| 3 | 30 | 15 | Incubate HRP anti-Rabbit Antibody |
| 4 | 30 | 30 | Color development - static for both |

What is claimed is:

1. In the process of performing heterogeneous immunoassays in microtiter plates having wells involving a period of substrate incubation time to capture antigens and/or label samples, the improvement comprising:
using a cylindrical rotator to create liquid rotation in the well during incubation time for antigen capture or sample labeling time in order to reduce the time of antigen capture and/or sample labeling.

2. The process in claim 1 where a microtiter vortexing instrument is used to create the cylindrical rotation in the well.

3. The process of claim 1 wherein the heterogeneous immunoassays is selected from the group consisting of scintillation counting, fluorescence, chemiluminescence, electrochemical assays and enzymatic methods, surface plasmon resonance, surface enhanced Raman scattering, quantum dots, and microcantilevers.

4. The process of claim 3 wherein the immunoassays is surface enhanced Raman scattering.

5. The process of claim 1 wherein total incubation time is reduced from 50% to 90% of that normally encountered without rotation during antigen binding and/or sample labeling (stagnant time).

6. The process of claim 1 wherein the total incubation time is reduced 95% of the stagnant times.

7. The process of claim 1 wherein rotation is at a speed sufficient to reduce incubation time for from 50% to 90%, but below the speed at which vortexing occurs.

8. The process of claim 1 wherein rotation occurs during both the time of antigen capture and sample labeling.

9. The process of claim 8 wherein the heterogeneous immunoassays is selected from the group consisting of scintillation counting, fluorescence, chemiluminescence, electrochemical assays and enzymatic methods, surface plasmon resonance, surface enhanced Raman scattering, quantum dots, and microcantilevers.

10. The process of claim 9 wherein the immunoassays is surface enhanced Raman scattering.

11. The process of claim 8 wherein total incubation time is reduced from 50% to 90% of that normally encountered without rotation during antigen binding and/or sample labeling (stagnant time).

12. The process of claim 8 wherein the total incubation time is reduced 95% of the stagnant times.

13. The process of claim 8 wherein rotation is at a speed sufficient to reduce incubation time for from 50% to 90%, but below the speed at which vortexing occurs.

14. A method of reducing assay time for heterogeneous immunoassays prepared by using an antigen binding step and thereafter a labeling step for the antigen antibody substrate, comprising:
rotating the substrate during incubation time for the antigen antibody mixture and during the labeling step.
